(19) United States
(12) Patent Application Publication
(10) Pub. No.: US 2026/0220781 A1
(43) Pub. Date: Jul. 30, 2026

(54) RADIOGRAPHIC IMAGE PROCESSING APPARATUS AND COMPUTER-READABLE RECORDING MEDIUM

(71) Applicant: KONICA MINOLTA, INC., Tokyo (JP)

(72) Inventors: Ryoichi WATANABE, Tokyo (JP); Kojiro ONO, Tokyo (JP); Kentaro OKAMOTO, Tokyo (JP)

(73) Assignee: KONICA MINOLTA, INC., Tokyo (JP)

(21) Appl. No.: 19/434,139

(22) Filed: Dec. 29, 2025

(30) Foreign Application Priority Data
Jan. 27, 2025 (JP) 2025-011196

(51) Int. Cl. G06T 7/00; A61B 6/00; A61B 6/50; G06T 7/20

(52) U.S. Cl. CPC G06T 7/0014; A61B 6/4405; A61B 6/503; G06T 7/20; G06T 2207/10116; G06T 2207/30048; G06T 2207/30061

(57) ABSTRACT

A radiographic image processing apparatus is a radiographic imaging processing apparatus capable of acquiring a dynamic image captured by radiographic imaging, and includes: a hardware processor that analyzes motion of at least one of a heart and a lung of a subject based on the dynamic image, in which the hardware processor infers, based on an analysis result, information on at least one of the heart and the lungs of the subject.

### CROSS REFERENCE TO RELATED APPLICATIONS

The entire disclosure of Japanese patent Application No. 2025 -011196, filed on January 27, 2025, is incorporated herein by reference in its entirety.

### BACKGROUND

### Technological Field

The present invention relates to a radiographic image processing apparatus and a computer-readable recording medium.

### Description of Related Art

Conventionally, in a radiographic imaging system, a system capable of capturing a dynamic image of a subject is known. For example, Japanese Unexamined Patent Publication No. 2019-5073 discloses a configuration in which an analysis result of a plurality of parts (e.g., the heart and the lungs) can be simultaneously displayed in a system capable of capturing a dynamic image.

### SUMMARY

Incidentally, it is known that diseases of the heart and the lungs are highly related to each other. Specifically, it is known that among diseases of one of the heart and the lungs, there are diseases for which the motion of the other of the heart and the lungs can serve as information for making a diagnosis.

However, conventionally, in a case of inspecting a disease of one of the heart and the lungs, it is common to inspect only one of the heart and the lungs. For example, in an echo examination or the like, only one of the heart and the lungs can be inspected, but the other of the heart and the lungs cannot be inspected at the same time. Accordingly, it has been difficult to grasp the synchronism between diseases of the heart and diseases of the lungs, and, when performing an examination that utilizes the relationship between the heart and the lungs, it has been necessary to carry out an additional examination of the other organ in addition to an examination of one organ, which has been inconvenient.

Objectives of the present invention include providing a radiographic image processing apparatus and a computer-readable recording medium capable of improving convenience in examination of at least one of the heart and the lungs.

In order to achieve at least one of the above-described objectives, a radiographic image processing apparatus reflecting one aspect of the present invention is

a radiographic imaging processing apparatus capable of acquiring a dynamic image captured by radiographic imaging, including:

a hardware processor that analyzes motion of at least one of a heart and a lung of a subject based on the dynamic image, wherein

the hardware processor infers, based on an analysis result, information on at least one of the heart and the lungs of the subject.

### BRIEF DESCRIPTION OF DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a diagram illustrating a radiographic imaging system according to an embodiment of the present invention;

FIG. 2 is a block diagram showing a configuration of a main part of the control system according to the present embodiment;

FIG. 3 is a block diagram illustrating an imaging controller;

FIG. 4 is a diagram illustrating a display example of an inference result; and

FIG. 5 is a flowchart illustrating an operation example of inference processing by an imaging controller.

### DETAILED DESCRIPTION OF EMBODIMENTS

Hereinafter, one or more embodiments of the present invention will be described with reference to the drawings. However, the scope of the invention is not limited to the disclosed embodiments.

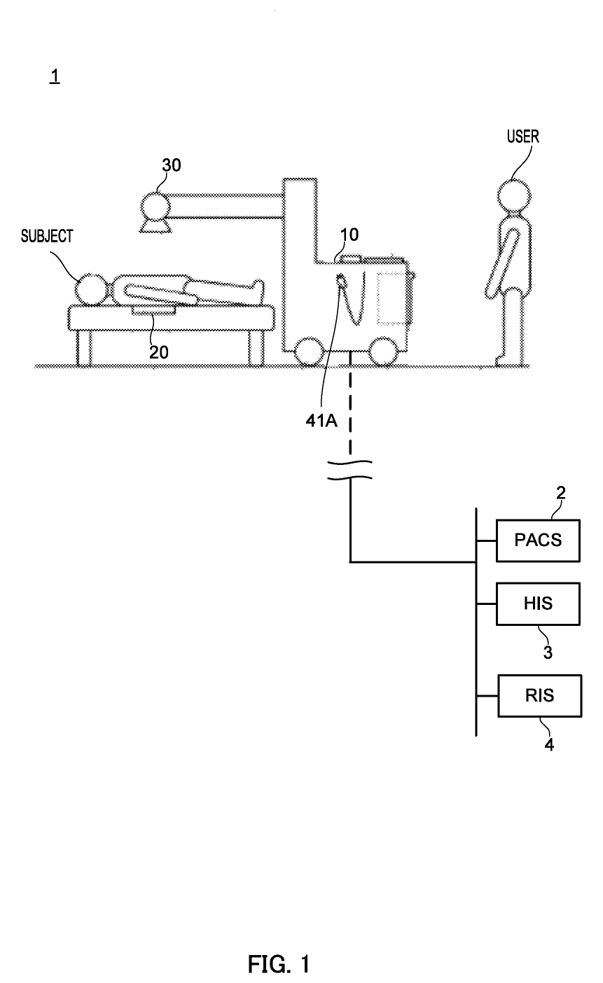

Hereinafter, an embodiment of the present invention will be described in detail with reference to the drawings. FIG. 1 is a diagram showing a radiographic imaging system according to an embodiment of the present invention.

As illustrated in FIG. 1, the radiographic imaging system 1 according to the present embodiment is capable of capturing still images and dynamic images, and is a round-visit (mobile) system for visiting a subject who is difficult to move and capturing radiographic images. Note that the radiographic imaging system 1 is applicable to, for example, a hospital room, an Intensive Care Unit (ICU), and an operating room.

The radiographic imaging system 1 includes a main body 10, one or more Flat Panel Detectors (FPDs) 20, and a radiation irradiation device 30.

Furthermore, an external system is connected to the radiographic imaging system 1 via a communication network. The external system is Picture Archiving and Communication Systems (PACS) 2, a Hospital Information Systems (HIS) 3, Radiology Information Systems (RIS) 4, and the like.

In the communication network including the radiographic imaging system 1, PACS 2, HIS 3, and RIS 4, for example, information is transmitted and received in accordance with a Digital Image and Communications in Medicine (DICOM) standard.

The FPD 20 is a portable radiographic detector that is capable of capturing still images and dynamic images. The FPD 20 includes, for example, radiation detection elements two dimensionally arranged on a glass substrate. The radiation detection elements are formed by semiconductor image sensors, such as photodiodes. The radiation detection elements detect, in accordance with the intensity of radiation, radiation that has been irradiated from the radiation irradiation device 30 (radiation source) and has passed through at least a subject, convert the detected radiation into electrical signals, and accumulate the electrical signals. For example, a switching unit such as a Thin Film Transistor (TFT) is connected to each radiation detection element. The switching unit controls the accumulation and reading of the electrical signals, and image data is acquired.

Note that the FPD 20 may be of an indirect conversion type in which radiation is converted into electrical signals by photoelectric conversion elements via a scintillator, or may be of a direct conversion type in which radiation is directly converted into electrical signals.

The FPD 20 is connected to the main body 10 of the radiographic imaging system 1 via a communication network, and performs wired communication or wireless communication with the main body. To be specific, the FPD 20 receives various control signals from the main body 10 and transmits generated image data to the main body 10 via the communication cable.

Furthermore, the main body 10 is provided with a storing part (not illustrated) capable of storing FPD 20. The main body 10 is carried to a hospital room in a state where the FPD 20 is stored in the storing part.

The radiation irradiation device 30 includes a synchronization signal output section (not illustrated), a generator (not illustrated), a radiation source (not illustrated), and the like.

The synchronization signal output section outputs a pulsed synchronization signal to each of the generator and the FPD 20 in response to operation of an exposure switch 41A, which will be described later. In the photographing of the still image, the synchronization signal is transmitted only once per still image, and in the photographing of the dynamic image, the synchronization signal of the same cycle is repeatedly transmitted a plurality of times.

The generator is configured to be able to apply, to the radiation source, a voltage corresponding to preset radiation irradiation conditions (a tube voltage, a tube current, an irradiation time, and the like) each time a synchronization signal is input from the synchronization signal output section.

The radiation source (tube) includes a rotary anode, a filament, and the like. The rotary anode is composed of, in addition to the target, a rotor or an anode rotor for rotating the anode, an anode shaft (anode axis), a bearing (not illustrated) and the like. The anode rotor rotates the target at a high speed by the principle of an induction motor. The filament is provided on a cathode sleeve disposed at a position facing the target. When a voltage is applied by the generator, the filament emits an electron beam corresponding to the voltage toward the rotary anode, and the rotary anode (anode rotor) generates a dose of radiation corresponding to the intensity of the electron beam.

In addition, the radiation source is rotated by a rotation shaft extending in the horizontal direction, and can be switched to a state in which an irradiation port of radiation is directed in the horizontal direction (a state in which three dimensional imaging is performed) or a state in which the irradiation port is directed in the vertical direction (a state in which imaging in a lying position is performed).

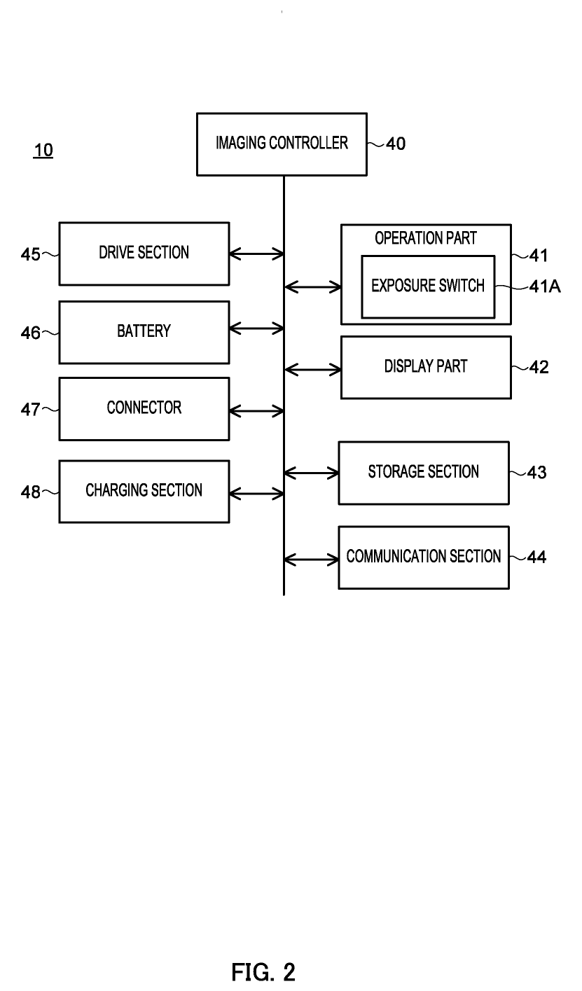

The main body 10 has a function as a console (radiographic imaging control apparatus). As shown in FIG. 2, the main body 10 includes an imaging controller 40, an operation part 41, a display part 42, a storage section 43, a communication section 44, a drive section 45, a battery 46, a connector 47, a charging part 48, and the like.

The imaging controller 40 is housed in the body 10 and includes a Central Processing Unit (CPU), a Read Only Memory (ROM), a Random Access Memory (RAM), and the like. In the imaging controller 40, the CPU reads a program corresponding to the processing details from the ROM and develops it in the RAM. Then, the imaging controller 40 cooperates with the developed program to centrally control the operation of each part.

Further, the imaging controller 40 analyzes the motion of at least one of the heart and the lungs of the subject based on the dynamic image, and infers information on at least the other of the heart and the lungs of the subject based

on the analysis result. The analysis and inference by the imaging controller 40 will be described later. The imaging controller 40 corresponds to the "radiographic image processing apparatus" according to the present invention.

The operation part 41 includes a touch screen or the like in which transparent electrodes are arranged in a lattice shape so as to cover the surface of the display part 42. The touch screen detects a position pressed with a finger, a touch pen, or the like, and inputs the position information as operation information to the imaging controller 40.

The operation part 41 includes an exposure switch 41A for operating the radiation irradiation device 30. The exposure switch 41A is a switch for a user to instruct radiation irradiation by the radiation irradiation device 30.

The display part 42 includes a monitor such as a Liquid Crystal Display (LCD) or a Cathode Ray Tube (CRT). The display part 42 performs display in accordance with an instruction of a display signal input from the imaging controller 40.

The storage section 43 is composed of a nonvolatile semiconductor memory, a hard disk and the like. The storage section 43 stores various programs executed by the imaging controller 40, parameters necessary for execution of processing by the programs, or data such as processing results.

Further, the storage section 43 is provided with a primary storage area (not illustrated) for temporarily storing the radiographic image transferred from the transfer unit FPD 20. Furthermore, the storage section 43 is provided with an image storage area (not illustrated) for storing the radiographic image transferred from the storage section FPD 20 in association with supplementary information for a certain period of time.

The communication section 44 transmits and receives data to and from the FPD 20 by wired communication or wireless communication. Furthermore, the communication section 44 transmits and receives data to and from an external device, such as the RIS and the PACS, via a network.

The drive section 45 is a circuit that drives a tube of a radiation source of the radiation irradiation device. The drive section 45 and the radiation source are connected to each other via a cable.

The battery 46 supplies electric power to each section of the body 10 and the radiation source. The battery 46 can be charged from the outside via an AC cable.

The connector 47 is provided inside the storing part and is electrically connected to the FPD 20 stored in the storing part.

The charging part 48 charges the FPD 20 connected via the connector 47 with electric power supplied from the battery 46 on the basis of the control by the imaging controller 40.

Next, analysis and inference performed by the imaging controller 40 will be described.

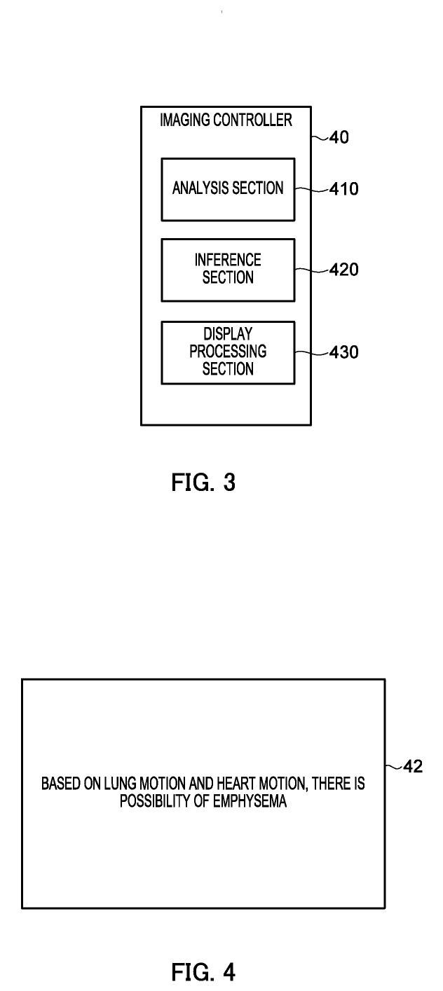

As illustrated in FIG. 3, the imaging controller 40 includes an analysis section 410, an inference section 420, and a display processing section 430.

It is known that diseases of the heart and the lungs are highly associated with each other. Specifically, it is known that among diseases of one of the heart and the lungs, there are diseases for which the movement of the other of the heart and the lungs can serve as information for making a diagnosis.

For example, in the case of emphysema, the elasticity of the lungs is lost, the alveoli are excessively expanded, and the compliance (flexibility) of the lungs is reduced. As a result, the load on the respiratory muscles increases and the respiratory muscles become fatigued, so that the decrease in the intrathoracic pressure during breathing becomes insufficient. As a result, venous return to the right ventricle of the heart (preload) decreases and blood inflow to the left ventricle of the heart (diastolic filling) also decreases.

As described above, in emphysema, blood inflow to the heart is reduced due to functional and structural changes of the lungs, and in particular, blood inflow in diastole is reduced. A decrease in the amount of blood flowing into the heart can be confirmed by, for example, a decrease in a change in signal value (concentration) when a region of interest is set in the left ventricle in a dynamic image of the heart, and therefore can be a basis for determination of emphysema.

In addition, when the valvular disease becomes severe, pulmonary congestion becomes strong. The degree of pulmonary congestion varies depending on the site. At a site with strong pulmonary congestion, the load on the respiratory organ increases, the change in the intrathoracic pressure increases, and the compliance of the lungs decreases. As a result, asymmetry occurs in the motion of the lungs. In this way, in the dynamic image of the lungs, the asymmetry of the motion of the lungs can be confirmed, and thus the dynamic image can be used as information for determining valvular disease.

That is, by analyzing the motion of at least one of the heart and the lungs of the subject on the basis of the dynamic images, information on at least the other of the heart and the lungs of the subject can be inferred.

The analysis section 410 analyzes the motion of both the heart and the lungs of the subject on the basis of the dynamic images acquired from the radiation irradiation device 30. The inference section 420 infers information on at least one of the heart and the lungs of the subject based on the analysis result of the analysis section 410. The information on the heart is information on heart disease, and the information on the lungs is information on lung disease.

For example, the analysis section 410 extracts a change in the signal value based on the motion of at least one of the heart and the lungs of the subject in the dynamic image. The signal value based on the motion is, for example, a signal value that can be measured in the region of interest set in the dynamic image, and is, for example, a value indicating the density of the image. Then, the inference section 420 infers information on at least one of the heart and the lungs of the subject on the basis of the change in the signal value.

For example, it is assumed that, for the motion of the heart in a dynamic image, a reduction in change in signal value when a region of interest is set in the left ventricle has been analyzed by the analysis section 410. In this case, the inference section 420 infers that the subject may have emphysema as the information on the lungs of the subject.

Further, for example, the analysis section 410 compares first image information based on the motion of at least one of the heart and the lungs of the subject in the dynamic image with second image information on the disease of at least the other of the heart and the lungs.

The first image information is an image of at least one of the heart and the lungs of the subject captured by the radiographic imaging system 1. The second image information is an image of at least the other of the heart and the lungs of another subject (a subject different from the subject of the first image information) having a disease of at least the other of the heart and the lungs.

Examples of a method of comparing the first image information and the second image information include a method of calculating a difference value between the first image information and the second image information, a method of calculating a similarity index of images, and a method of comparing histograms of images. In addition, examples of the method of comparing the first image information and the second image information also include a method of comparing frequency components of the images, a method of comparing image quality information, a method of comparing distances of feature vectors in the images, and a method of comparing segment shapes or color distributions by region division in the images. The method of comparing the first image information and the second image information may be other known methods.

For example, it is assumed that there is asymmetry in the motion of the lungs in the dynamic image, the first image information and the second image information are compared by the analysis section 410, and the difference therebetween is equal to or less than a predetermined threshold value. In this case, the inference section 420 infers that there is a possibility that the subject has valvular disease as the information on the heart of the subject.

In this way, the motion of at least one of the heart and the lungs of the subject is analyzed to infer information on at least the other of the heart and the lungs of the subject. As a result, the high relevance between the heart and the lungs can be effectively utilized.

Note that each of the information on the heart of the subject and the information on the lungs of the subject may be described in, for example, a table or the like in which various conditions and diseases are associated with each other. For example, in the above example, the condition that the change in the signal value of the left ventricle in the motion of the heart decreases is associated with emphysema, and the condition that the difference between the first image information and the second image information is equal to or smaller than a predetermined threshold value is associated with valvular disease. The inference section 420 refers to the above-described table and sets a disease for which the condition of the analysis result matches as information on the heart and the lung of the subject. Note that in addition to the table, each of the information on the heart of the subject and the information on the lungs of the subject may be described in a vector, a matrix, a data frame, a list, an array, a database, or the like.

Furthermore, the inference section 420 may infer information on one of the heart and the lungs of the subject on the basis of an analysis result of the one of the heart and the lungs of the subject and infer information on the one of the heart and the lungs of the subject on the basis of an analysis result of the other of the heart and the lungs of the subject.

For example, it is assumed that the inference section 420 infers, based on the motion of the lungs in the dynamic image, that the subject may have emphysema. Next, when the inference section 420 infers that the subject may have emphysema based on the signal value based on the motion of the heart in the dynamic image, it is inferred that the subject is highly likely to have emphysema from the viewpoint of both the lungs and the heart.

Furthermore, it is assumed that, based on the motion of the heart in the dynamic image, the inference section 420 infers that the subject has a possibility of valvular disease. Then, if the inference section 420 infers that the subject has a possibility of valvular disease on the basis of the motion of the lungs in the dynamic image, it is inferred that the subject has a high possibility of valvular disease from the viewpoints of both the lungs and the heart.

Thus, not only the inference result of one of the heart and the lungs but also the inference result of the other of the heart and the lungs can be used as a material for determining the disease of the one of the heart and the lungs of the subject.

For example, it is assumed that, in a case where determination is made based on only the motion of any one of the heart and the lungs of the subject, an opinion is presented that there is a possibility of a predetermined disease. However, a possibility of another disease cannot be denied in such a case where the signal value becomes low or a case where the white area becomes large in the dynamic image, and thus the validity of the above-described opinion may be insufficient.

In contrast, adding not only the inference result (primary determination) of one of the heart and the lungs of the subject but also the inference result of the other as materials for determination can promote the primary determination of the disease. That is, it is possible to improve the validity of the opinion based on the diagnosis. Furthermore, in a case where one inference result is different from the other inference result, it is possible to make it easier to consider recommendation of an examination by another modality.

Further, the inference section 420 may infer information on both the heart and the lungs of the subject based on the analysis result of the heart of the subject, and may infer information on both the heart and the lungs of the subject based on the analysis result of the lungs of the subject.

As a result, inference results of both the heart and the lungs (two inference results) are obtained from the analysis result of the heart, and inference results of both the heart and the lungs (two inference results) are obtained from the analysis result of the lungs. That is, since four inference results are obtained by the analysis of the heart and the lungs, it is possible to easily suppress the occurrence of a failure to diagnose a disease or the like.

Furthermore, the inference section 420 may infer information on each of the heart and the lungs of the subject, based on the analysis result of one of the heart and the lungs of the subject by the analysis section 410.

For example, it is assumed that a lung of a subject has been diagnosed. At this time, if the inference section 420 can infer information on not only the lungs but also the heart, it is possible to indicate to the subject that not only the lungs but also the heart may have a disease. As a result, it is possible to suppress a problem such as a delay in treatment due to a diagnosis failure.

Incidentally, in a dynamic image, it may be difficult to check the motion of one of the heart and the lungs. For example, in a case where it is difficult to visually recognize the lung field in the dynamic image of the motion of the lungs, there is a possibility that appropriate diagnosis cannot be performed with the dynamic image of the motion of the lungs. In this case, in the dynamic image in the motion of the heart, the inference section 420 infers the information on the lungs, whereby the minimum information on the lungs can be obtained.

Furthermore, assume that a predetermined disease has been diagnosed by another modality. For example, it is assumed that a diagnostic result indicating that there is a possibility of a predetermined disease in the heart has been obtained in echocardiography. In addition, the radiographic imaging system 1 can support diagnosis in another modality by obtaining an inference result of information on the heart based on the motion of both the heart and the lungs.

Furthermore, the inference result of the inference section 420 is output to a display medium (e.g., the display part 42) by the display processing section 430. That is, the display processing section 430 outputs information based on the inference result of the inference section 420 to the display part 42.

For example, when it is inferred that there is a possibility of emphysema from both of the motion of the lungs and the motion of the heart, information indicating that fact is displayed on the display part 42. In FIG. 4, an example is illustrated in which "BASED ON LUNG MOTION AND HEART MOTION, THERE IS POSSIBILITY OF EMPHYSEMA" is displayed. In addition, when there is score information indicating the degree of possibility of a disease, the score information may be displayed on the display part 42. In this case, for example, a display such as “BASED ON LUNG MOTION AND HEART MOTION, SCORE OF EMPHYSEMA IS 60 " is displayed on the display part 42.

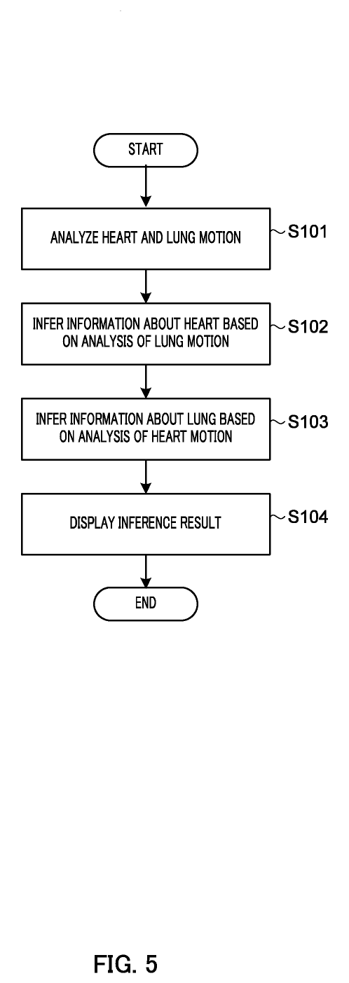

Next, a flow of processing performed by the imaging controller 40 will be described. FIG. 5 is a flowchart illustrating an operation example of inference processing by the imaging controller 40. This control is appropriately executed in the radiographic imaging system 1 when the imaging controller 40 acquires a captured radiographic image (dynamic image).

As illustrated in FIG. 5, the imaging controller 40 analyzes the heart and the lungs based on the dynamic image (step S101). After step S101, the imaging controller 40 infers information on the heart by analyzing the motion of the lungs (step S102), and infers information on the lungs by analyzing the motion of the heart (step S103).

Note that in step S102, the imaging controller 40 may infer information on the lungs together with information on the heart by analyzing the motion of the lungs. Furthermore, in step S103, the imaging controller 40 may infer information on the heart together with information on the lungs by analyzing the motion of the heart. Further, in step S102, the imaging controller 40 may infer the information on the heart by analyzing the motion of the heart in addition to the analysis of the motion of the lungs. Further, in step S103, the imaging controller 40 may infer the information on the lungs by analyzing the motion of the lungs in addition to the analysis of the motion of the heart. Furthermore, one of step S102 and step S103 may be performed, and the other of step S102 and step S103 may not be performed.

After step S103, the imaging controller 40 displays the inference result (step S104). After step S104, the present control ends.

According to the present embodiment configured as described above, based on the analysis result of the motion of at least one of the heart and the lungs of the subject, information on at least the other of the heart and the lungs of the subject is inferred. As a result, the high relevance between the heart and the lungs can be effectively utilized, and the convenience in the examination of at least one of the heart and the lungs can be improved.

Further, the information on one of the heart and the lungs of the subject is inferred based on the analysis result of the one of the heart and the lungs of the subject, and the information on one of the heart and the lungs of the subject is inferred based on the analysis result of the other of the heart and the lungs of the subject.

As a result, not only the inference result of one of the heart and the lungs but also the inference result of the other of the heart and the lungs can be used as materials for determining the disease of the one of the heart and the lungs of the subject, thus promoting the determination of the disease. That is, it is possible to improve the validity of the opinion based on the diagnosis. Furthermore, in a case where one inference result is different from the other inference result, it is possible to make it easier to consider recommendation of an examination by another modality.

In addition, information on both the heart and the lungs of the subject is inferred based on the analysis result of one of the heart and the lungs of the subject, and information on both the heart and the lungs of the subject is inferred based on the analysis result of the other of the heart and the lungs of the subject.

As a result, both inference results of the heart and the lungs (two inference results) are obtained from the analysis result of the heart, and both inference results of the heart and the lungs (two inference results) are obtained from the analysis result of the lungs. That is, since four inference results are obtained by the analysis of the heart and the lungs, it is possible to easily suppress the occurrence of a failure to diagnose a disease or the like.

In addition, since the information on each of the heart and the lungs of the subject is inferred based on the analysis result of the motion of any one of the heart and the lungs of the subject, it is possible to indicate to the subject that, for example, not only the lungs but also the heart may have a disease. As a result, it is possible to suppress a problem such as a delay in treatment due to a diagnosis failure.

The imaging controller 40 is provided in the mobile radiographic imaging system 1. That is, in the present embodiment, a radiographic image can be captured after the radiographic imaging system 1 is moved to the place of the subject.

Incidentally, in postoperative patients in intensive care or the like, it is required to grasp the pathological conditions of emphysema, atelectasis, pneumonia, pleural effusion, pneumothorax, and the like and take appropriate therapeutic intervention for each of them. In such a pathological condition, diagnosis by Computed Tomography (CT) is ideal. However, since there are patients, for example, seriously injured patients, who are difficult to move to a CT room, there is a possibility that appropriate treatment cannot be performed. In addition, since the CT room may be occupied by a reservation, there is a possibility that imaging cannot be performed at an appropriate timing.

In the present embodiment, since the imaging controller 40 is provided in the mobile radiographic imaging system 1, the system can be moved for imaging to the place of the patient who is difficult to move to the CT room. As a result, appropriate treatment can be performed. In addition, since it is possible to move to the patient and quickly perform imaging, it is possible to perform imaging at an appropriate timing.

Note that although the motion of both the heart and the lungs of the subject is analyzed in the above-described embodiment, the present invention is not limited thereto, and for example, a configuration may be adopted in which the motion of only one of the heart and the lungs of the subject is analyzed. Further, in this case, the inference section 420 may be configured to infer information on only one of the heart and the lungs of the subject based on the analysis result of the motion of the other of the heart and the lungs of the subject.

Although emphysema and valvular disease are given as examples of diseases indicating a high relevance between the heart and the lungs in the above embodiment, the present invention is not limited thereto. For example, the disease includes pulmonary hypertension, pulmonary embolism, heart failure and the like.

In the case of pulmonary hypertension, blood vessel remodeling occurs in which the blood vessel wall of the pulmonary artery thickens and hardens. Due to this vascular remodeling, the extensibility of the blood vessel decreases, and the compliance decreases. As a result, the expandability of the blood vessel is reduced, and thus the change in the diameter of the blood vessel during diastole in the heart is reduced. This can be confirmed by, for example, a decrease in signal value change when a region of interest is set in the pulmonary artery in the heart, and thus can be a material for determining pulmonary hypertension.

In the case of a pulmonary embolism, a loss of signal value change in the peripheral vascular region occurs due to a thrombus in the pulmonary blood vessel. Then, the heart starts to hypercontract in order to perform functional compensation. That is, by confirming the hypercontractility of the heart, the examination of the heart can be diagnostic reference information for pulmonary embolism.

In the case of heart failure (in particular, right heart failure), the force of feeding blood to the lungs decreases, so that blood stays in the pulmonary vessels to cause pulmonary congestion. Therefore, by confirming pulmonary congestion, examination of the lungs can be diagnostic reference information for heart failure.

In the above-described embodiment, the radiographic imaging control apparatus (radiographic image processing apparatus) is the radiographic imaging system 1 including the radiation irradiation apparatus 30, but the present invention is not limited thereto. For example, a radiographic imaging control apparatus separate from the radiation irradiation apparatus may be arranged in an operation room separate from an imaging room in which the radiation irradiation apparatus is arranged.

In addition, each of the above-described embodiments is merely an example for embodying the present invention, and the technical scope of the present invention should not be interpreted in a limited manner by these embodiments. That is, the present invention can be implemented in various forms without departing from the spirit or main features thereof.

Although embodiments of the present invention have been described and illustrated in detail, the disclosed embodiments are made for purposes of illustration and example only and not limitation. The scope of the present invention should be interpreted by terms of the appended claims.

What is claimed is:

1. A radiographic imaging processing apparatus capable of acquiring a dynamic image captured by radiographic imaging, the radiographic imaging processing apparatus comprising:
a hardware processor that analyzes motion of at least one of a heart and a lung of a subject based on the dynamic image, wherein
the hardware processor infers, based on an analysis result, information on at least one of the heart and the lungs of the subject.

2. The radiographic image processing apparatus according to claim 1, wherein
the hardware processor is configured to:
analyze motion of both the heart and the lung of the subject based on the dynamic image, and
infer information on one of the heart and the lung of the subject based on the analysis result of the one of the heart and the lung of the subject, and infer the information on the one based on the analysis result of the other of the heart and the lung of the subject.

3. The radiographic image processing apparatus according to claim 1, wherein
the hardware processor infers information on both the heart and the lung of the subject based on the analysis result of one of the heart and the lung of the subject, and infers the information on both the heart and the lung of the subject based on the analysis result of the other of the heart and the lung of the subject.

4. The radiographic image processing apparatus according to claim 1, wherein
the hardware processor is configured to:
analyze motion of one of the heart and the lung of the subject based on the dynamic image, and
infer information on each of the heart and lung of the subject based on the analysis result.

5. The radiographic image processing apparatus according to claim 1, wherein
the hardware processor is configured to:
extract a change in signal value based on the motion of the at least one of the heart and the lung of the subject in the dynamic image, and
infer information on at least the other of the heart and the lung of the subject based on the change in signal value.

6. The radiographic image processing apparatus according to claim 1, wherein
the hardware processor is configured to:
compare first image information based on the motion of the at least one of the heart and the lung of the subject in the dynamic image with second image information on a disease of at least the other of the heart and the lung; and
infer information on at least the other of the heart and the lung of the subject based on a result of comparison between the first image information and the second image information.

7. The radiographic image processing apparatus according to claim 1, wherein
the radiographic image processing apparatus is provided in a mobile radiographic imaging system.

8. A computer-readable recording medium storing therein a program for a radiographic image processing apparatus capable of acquiring a dynamic image captured by radiographic imaging, the program causing a computer to execute:
analysis processing of analyzing motion of at least one of a heart and a lung of a subject based on the dynamic image, and
inference processing of inferring information on at least the other of the heart and the lung of the subject based on an analysis result of the analysis processing.
